(19) United States
(12) Patent Application Publication
(10) Pub. No.: US 2011/0202019 A1
(43) Pub. Date: Aug. 18, 2011

(54) HAND HELD SKIN TREATMENT SPRAY SYSTEM WITH AIR HEATING ELEMENT

(75) Inventors: Steven C. Cooper, Athens, GA (US); Scott Thomason, Macedonia, OH (US); Louis J. Monaco, South Euclid, OH (US)

(73) Assignee: MT Industries, Inc., Macedonia, OH (US)

(21) Appl. No.: 13/091,337

(22) Filed: Apr. 21, 2011

Related U.S. Application Data
(63) Continuation-in-part of application No. 12/910,754, filed on Oct. 22, 2010.
(60) Provisional application No. 61/266,810, filed on Dec. 4, 2009.

(51) Int. Cl. A61M 35/00

(52) U.S. Cl. 604/291

(57) ABSTRACT

A hand held spray system for skin treatments includes air outlets for delivering heated air to improve the efficacy and comfort of the spraying experience. The heated air may also heat a liquid tip and transfer this heat to the skin treatment. The heated air may be applied while spray is emitted from the nozzle to increase the spray cloud temperature, or may be applied before or after the spray application, with the spray turned off, to warm or dry the skin. The spray system includes a heating source to warm the air as it passes through the system and is delivered through atomizing and/or pattern shaping orifices.

### PRIORITY CLAIM

This application is a continuation-in-part of prior U.S. patent application Ser. No. 12/910,754, filed on Oct. 22, 2010, which claims priority from U.S. Provisional Application for Patent No. 61/266,810, filed Dec. 4, 2009, the disclosures of both of which are hereby incorporated by reference.

### BACKGROUND

Spray devices for the application of liquids onto human skin and hair are well known. Sprays are used for many types of medicines, skin treatments, hair treatments, deodorants, lotions, and cosmetic agents. Specialized hand-held and automated spray systems are used in tanning salons and spa treatment centers to apply sunless tanning compounds and skin care formulas, such as moisturizers, anti-aging treatments, and exfoliants. The spray solution used for sunless tanning is generally a water-based mixture of DHA (dihydroxyacetone) and/or erythrulose and various other skin care ingredients such as aloe vera. Often a cosmetic bronzer is added along with pleasant scents and ingredients to enhance tanning performance, such as formulations to balance skin ph. For best results, the spraying of the solution utilizes a finely atomized spray (mist), as opposed to the use of a spray stream or large spray droplets, because the mist of solution provides for even coverage and reduces the risk of streaking or running of the spray deposit.

The skin treatment spray process has inherently been a cold, uncomfortable experience for the recipient as nozzle expansion effects significantly cool the air and liquid in the spray cloud during application to the skin. Furthermore, cold skin is known to inhibit optimum absorption of the skin care ingredients. Temperatures of the spray cloud can be over 30 degrees (F.) lower than human body temperature and significantly cooler than ambient temperature (of the liquid or the air emitted from the sprayer).

In salons, customers disrobe for the spray treatment which lasts from 30 seconds to 5 minutes. Some treatments involve sequential spray regimens of alternate ingredients so the experience can be significantly longer. Thus, the length of time the customer is exposed to cold can be significant and may discourage the customer from obtaining the treatment in the first place or returning for an additional treatment at a later date.

Moreover, “goose bumps” or “chill bumps” may form on the skin as an involuntary pilomotor reflex reacting to receiving a cold spray. Applying a spray tanning treatment to skin with chill bumps often produces a poor result. One reason for the poor result is an uneven formation of the chill bumps on certain parts of the body but not on others. For example, chill bumps are more likely to form on a subject's forearm than underneath the arm. Also, chill bumps are more pronounced on a subject's chest than on the subject's stomach; they are also more pronounced on a subject's thighs than on the calves. The resulting tan will be different when a spray tan is applied to a body part with chill bumps than will result when applied to a body part without chill bumps. Often, the resulting tan may have an uneven tan color and uneven tanning spray penetration of the skin layers. The chill bumps may also contribute to increased beading, which is the formation of collected and coalesced droplets of spray tanning solution on the skin and hairs. This beading may cause undesirable “freckling” effects.

After the spray treatment, customers often use a towel to dry their skin. The action of toweling-off removes a significant quantity of the sprayed ingredients from the skin. The remaining ingredients may be redistributed, which can produce a splotchy appearance in the case of sunless tanning or other cosmetic treatments. If the customer opts not to use a towel, and instead simply dry off in the ambient air, the surface of the skin can become sticky.

Many tanning salons providing the new sunless spray tanning service also have conventional UV lamp tanning beds. Customers have observed that application of sunless tanning solutions quickly after they use a UV tanning bed can result in a deeper and darker DHA tan. It is important to move from the UV tanning bed to receive a spray of sunless tanning solution as quickly as possible. It is also essential to remove all perspiration resulting from the UV treatment or the tan result can be uneven. The benefits of UV tanning coupled with a sunless tanning spray may be due to opening the pores of the skin and from more thoroughly and more deeply drying out of the top skin layer by the hot UV lamps. However, due to skin health concerns, many customers do not wish to use the UV beds and therefore cannot take advantage of this practice to enhance their sunless tan.

DHA tans the skin by reacting with proteins in the stratum corneum, the top protective skin layer composed of dead skin cells. It is known that only the uppermost dry layers of the stratum corneum will tan effectively with DHA or erythrulose. Very dry skin will pigment the darkest and layers containing surface moisture will not tan nearly as well. Skin care specialists suggest using a warm towel on the skin before application of spray treatments since warm skin may better absorb some ingredients. However, a skin surface that is too hot will perspire, thus reducing the effectiveness of the sprayed ingredients.

A need exists in the art to address the foregoing issues in connection with providing a better sunless tanning experience and result for the consumer.

Reference is further made to Venuto, U.S. Pat. No. 6,554,208 (the disclosure of which is hereby incorporated by reference) which teaches a tanning spray booth implementation with a nozzle operable to both spray tanning solution and deliver drying air when not spraying.

Reference is also made to Safara, U.S. Pat. No. 5,991,937 (the disclosure of which is hereby incorporated by reference) which teaches a bidet sprayer implementation operable to both spray cleaning water streams and deliver drying air when not spraying.

### SUMMARY

Embodiments disclosed herein propose the controlled application of warm air and warm liquid in connection with applications of atomized (misted) sunless tanning sprays using a hand held type of spray system. This controlled application enhances efficacy of the tanning compounds and results in a deeper tan color and a longer lasting tan. In addition, the mixing of heated air and heated liquid into the atomized spray cloud reduces the discomfort caused by the inherently cold spray stream. Furthermore, warm air and liquid enhances the spray uniformity result and produces a softer characteristic feel of the spray ingredients on the skin, while reducing complaints of “stickiness” or “tackiness” by the consumer. Deposition efficiency and uniformity of the tan result is also improved.

A spray nozzle system in a hand held spray format is presented for applying topical skin treatments, such as sunless tanning formulations, medicines, and lotions. Specifically, liquids or suspensions are applied to human skin using a hand held spray system which allows for controlled operation of a heated air system and a heated atomizing spray liquid dispensing system.

A spray nozzle system includes an air outlet or outlets positioned near the liquid spray outlet of the spray nozzle to deliver heated air and heated liquid to improve the atomization of the spray and the comfort of the spraying experience. The heated air may be applied to atomize or shape the spray cloud that is emitted from the nozzle to increase the spray cloud temperature, or may be applied before or after the spray application, with the spray turned off, to warm or dry the skin.

The handheld spray device includes at least one air pathway containing a heating element; the air path terminates at an air assisted or an air-atomizing spray nozzle system. The nozzle may be of any type of air-assisted nozzle or air-atomizer known in the art, with or without pattern shaping jets, and with or without adjustable porting allowing control of pattern shaping jets. High volume, low pressure (HVLP), low volume, low pressure (LVLP), and adjustable volume, adjustable pressure (AVAP) are types of air atomizing nozzles that may be used with the disclosed spray gun. Other types of spray nozzles may also be used, such as air-assisted, hydraulic, and airbrush nozzles. Spraying systems according to embodiments of the present disclosure may be particularly suited for coating a target surface because the spray nozzle is capable of producing a well atomized, defined, and shaped spray pattern.

The heating element is positioned within the spray gun upstream of and close to the point of atomization, which provides warmer air and eliminates the disadvantages of a heavy, insulated hose in the event the heating element is located at the source of compressed air. As the heated air flows through the spray gun, it also heats a thermally conductive liquid tip, which in turn warms the liquid flowing through the tip. Heated air and liquid that is emitted from the spray gun may improve spray atomization and create a more comfortable spray tanning experience. Also, warmed liquid flowing through the liquid channels of the spray gun may be less resistant to collection in the liquid channels, which may make the spray gun easier to clean and maintain.

The heating element may be constructed of resistive heating wire and may be contained within the spray gun handle or any other section of the spray gun. The heating element may be insulated to provide both thermal and electrical protection between the heating element and the hand of the operator.

In another embodiment, the heated airflow is redirected from the nozzle jets to one or more of the supplemental air outlets. In this embodiment, a control valve may be used to proportion the amount of airflow directed to the main atomizer air jets, the pattern shaping air jets, and any other air outlets of the spray gun.

The method of applying heated air in connection with layered applications of atomized spray deposition has been found to make the experience of skin spray treatments much more comfortable as well as improve coating uniformity. In addition, this method provides an improved tack-free feel of the spray deposit on the skin both during and after the spray session. In the case of sunless tanning with active ingredients such as Erythrulose or DHA (dihydroxyacetone), the system provides for an improved tanning color and increased longevity of the tan.

### BRIEF DESCRIPTION OF THE DRAWINGS

A more complete understanding of the invention may be obtained by reference to the following drawings:

FIG. 1 schematically illustrates a spraying system adapted for use in hand held spraying application;

FIGS. 2A and 2B illustrate coating spray patterns that may be delivered by a hand held spraying application according to embodiments of the present disclosure;

FIG. 3 shows additional schematic detail of the base unit shown in FIG. 1;

FIG. 4 illustrates an exemplary implementation of a hand held sprayer of the type shown in FIG. 1;

FIG. 5 illustrates an exemplary implementation of a hand held sprayer of the type shown in FIG. 1;

FIG. 6 illustrates a cross sectional view of the hand held sprayer shown in FIG. 4;

FIGS. 7A and 7B illustrate embodiments of a nozzle that may be used with the hand held sprayer of the present disclosure;

FIGS. 8A and 8B illustrate embodiments of keyed connectors that may be used with the hand held sprayer of the present disclosure;

FIGS. 9A to 9C illustrate various views of an air heating system used within the hand held sprayer of the present disclosure;

FIG. 10 illustrates a graph of heater operation in multiple modes; and

FIG. 11 illustrates a graph of temperature of a liquid tip used in a hand held spraying operation.

### DETAILED DESCRIPTION OF THE DRAWINGS

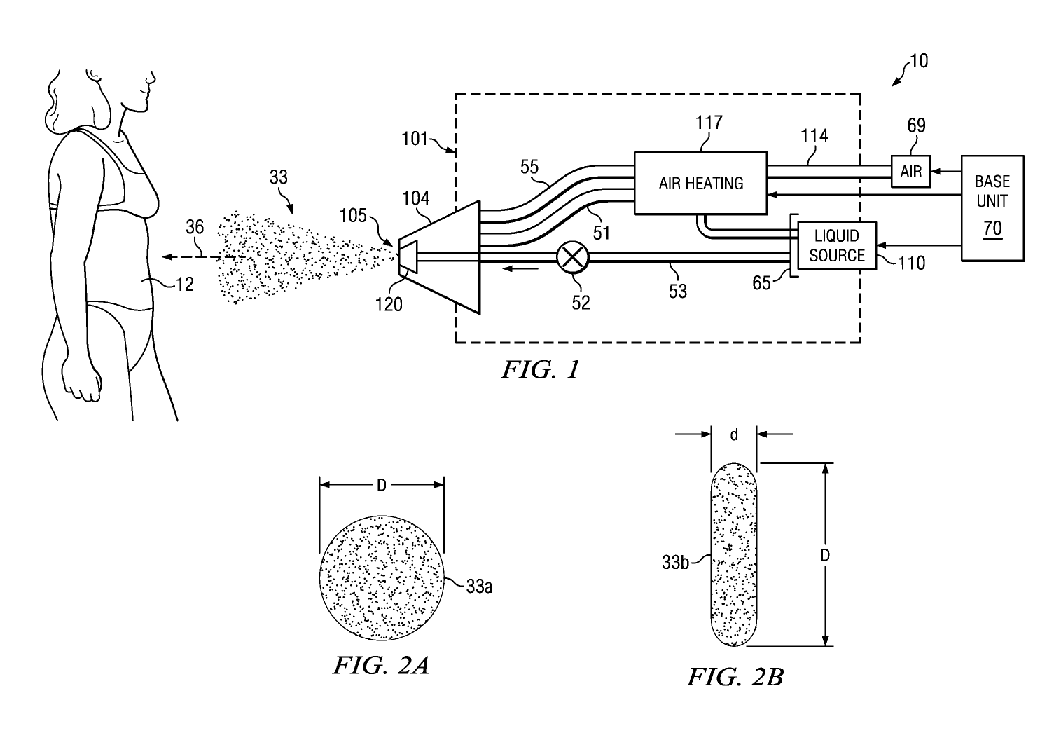

Reference is now made to FIG. 1 which schematically illustrates a spraying system 10 adapted for use, for example, in a hand held spraying application. The system 10 is configured to apply an atomized mist of warmed skin treatment liquid and warmed air towards a target surface 12 (for example, a customer's skin). The system 10 comprises a hand held spray member (in this case schematically represented by a dotted enclosing line 101, wherein the enclosing line 101 for the spray member generally indicates the use of any suitable enclosure or housing configuration including, for example, a simple structural mount to which spray member components are mounted or a casing which completely encapsulates the spray member components). The line 101 thus generally represents the support, enclosure or housing configuration of the hand held spray member.

Supported by the support, enclosure or housing configuration 101 of the spray member is a nozzle 104 that includes a spray jet outlet 105. The spray jet outlet 105 of the nozzle 104 emits warm air and warm liquid from separate orifices to create a finely atomized spray cloud (for example, a mist cloud) 33 of the skin treatment liquid aimed generally in a spray direction 36.

The nozzle 104 with spray jet outlet 105 may comprise any suitable finely atomizing spray nozzle assembly known to those skilled in the art. For example, the nozzle 104 may comprise any known air-atomizing type atomizing nozzle, such as a high volume, low pressure (HVLP) nozzle, a low volume, low pressure (LVLP) nozzle, or an adjustable volume, adjustable pressure (AVAP) nozzle. In certain embodiments, the nozzle 104 may not be an air-atomizing nozzle, but rather may be a hydraulic nozzle, a sonic nozzle, or any other nozzle that uses air in connection with creating a spray that may be used for coating a target surface.

In the case of an air-atomizing nozzle, an air source may be used by the nozzle 104 to atomize the spray liquid and form the spray cloud 33 (as well as air used by the nozzle 104 to shape the pattern of the emitted spray cloud). In the case of a mechanical, sonic, or hydraulic air atomizer, air may not directly cause the atomization of the spray, but instead may be used for spray delivery, turbulent flow formation, pattern shaping, or directional spray control. The air may be heated by a heating element before reaching the nozzle 104. The nozzle 104 may also include a liquid tip body 120 through which liquid flows. The liquid tip body 120 may be surrounded by an internal heated air stream. In certain embodiments, the nozzle 104 may also support electrostatic spraying of the skin treatment liquid that may have been heated by the internal air stream.

A liquid inlet 53 may be coupled to a liquid source 110 at one end and coupled to a liquid tip body 120 at another end. The liquid flow may be controlled in certain embodiments by a valve 52, pump, or other liquid flow control device that may be located along the liquid path defined by the ducting of the liquid inlet 53. The liquid tip body 120 may be associated with the nozzle 104, and it may receive the liquid before it is emitted as part of the mist 33. In addition, the liquid tip body 120 may be heated by the heated air flowing through the nozzle 104. The heated liquid tip body 120 may in turn heat the liquid that flows through the liquid tip body 120. In addition to providing a more comfortable feel on the skin, the heated liquid may also flow through the liquid tip body 120 more easily. It may also create an improved atomized mist where spray droplets may be a more uniform size. Thus, an improved spray pattern of mist 33 may be received by the target surface 12.

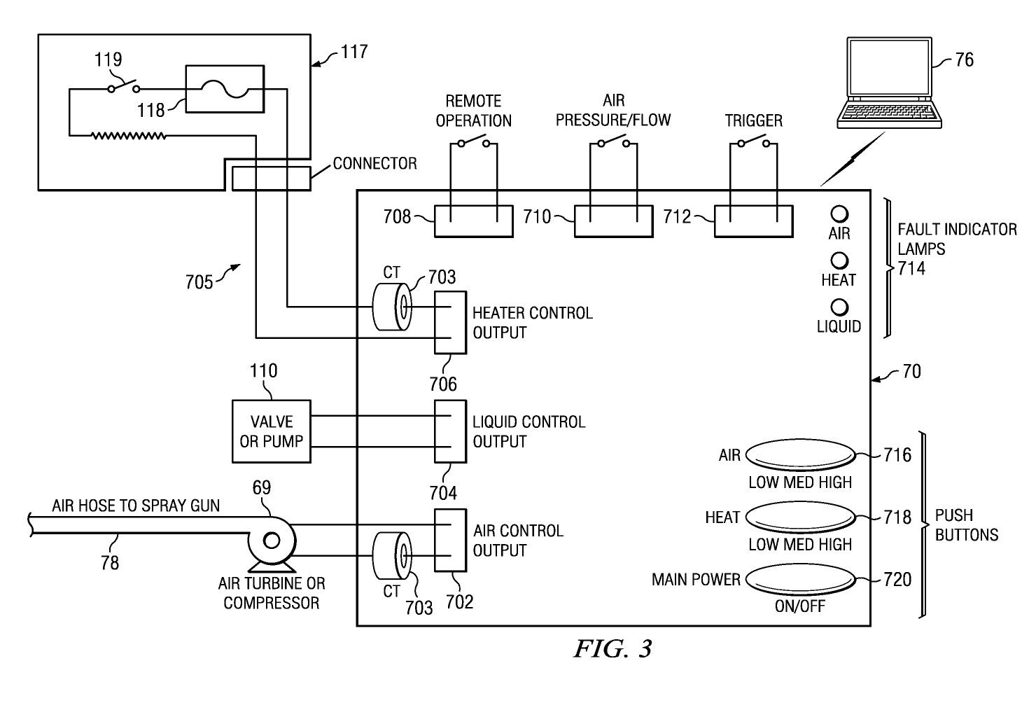

The spray cloud 33 may be a variety of shapes. For example, as shown in FIG. 1, the spray cloud may be a diverging spray pattern, which may be known as a flat, fan spray pattern. When the hand held spray member 101 is held approximately four to 18 inches away from the target surface 12, a particularly shaped coverage area may result. An example of a coverage area of the flat fan spray pattern 33b is shown in FIG. 2B. Flat fan spray pattern 33b may be generally elliptical in shape. It may have a major diameter (D) that is approximately one to six inches in length. Its minor diameter (d) may be in the range of 1/10 to ½ the major diameter D. A coverage area for a solid cone spray pattern 33a is shown in FIG. 2A. The diameter of the pattern 33a may be between 0.1 and five inches. The present disclosure is not limited to forming only these two spray patterns, but rather a hollow cone spray pattern may also be created according to the teaching of the present disclosure. In certain embodiments, as explained in greater detail below, the hand held spray member 101 may include pattern shaping jets, which may be used to create the different spray patterns.

The liquid source 110 may be a container that is filled with the skin treatment liquid. That container may be an integral component of, or may be removably mounted to, the support, enclosure or housing configuration 101 of the hand held spray member. The container may be sized to store a relatively small amount of skin treatment liquid (for example, one or a few doses selected for each spray session or application). The container may be received by a receptacle 65 formed in the support, enclosure or housing configuration 101 of the hand held spray member and coupled to the liquid inlet 53. In an alternative configuration, the container may instead comprise an external tank configuration storing the skin treatment liquid and coupled to the liquid inlet 53 using a hose.

The reference to a liquid source 110 includes the use of a single liquid tank supplying a single type (or container) of liquid for spray application as well as the use of multiple liquid tanks (or containers) each containing a distinct liquid for customer selection and skin application. When multiple tanks are provided, the customer can design a multi-product spray session. The operation of the system 10 can be adapted to optimize the spray experience based on the liquid selections made by the customer. Selection may be made by the user between different spray liquid products, such as moisturizers, sunless tanning products, and certain skin conditioning compounds known to improve the efficacy of sunless tanning products.

Further supported by the support, enclosure or housing configuration 101 of the hand held spray member is an air heating system 117 coupled to supply heated air to the nozzle 104. The air heating system 117 receives air from inlet air ducting 114 and heats the air to a higher temperature than the temperature of the air as received. Any suitable heating element could be used within the air heating system 117. Air heated by the air heating system 117 may flow through main air ducting 51 to the nozzle 104. Depending on the type of nozzle, the heated air may help atomize the mist 33, dry the target surface 12, and the like. Air heated by the air heating system 117 may travel through a separate pattern shaping air duct to nozzle 104. This air may be emitted through a pattern shaping orifice and help shape the pattern of the spray mist 33 to allow it to effectively coat the target surface 112. In certain embodiments, the heated air may also flow through the spray member 101 and through a check valve and heat and/or pressurize liquid in the liquid source 110.

The heating element may receive power from a power supply that is either internal or external to the hand held spray member, such as base unit 70. The heating element for the air heating system 117 can be incorporated directly into inlet air ducting 114. In a preferred implementation, as discussed in more detail herein, the heating element for the air heating system 117 is positioned in a handle of the hand held spray member.

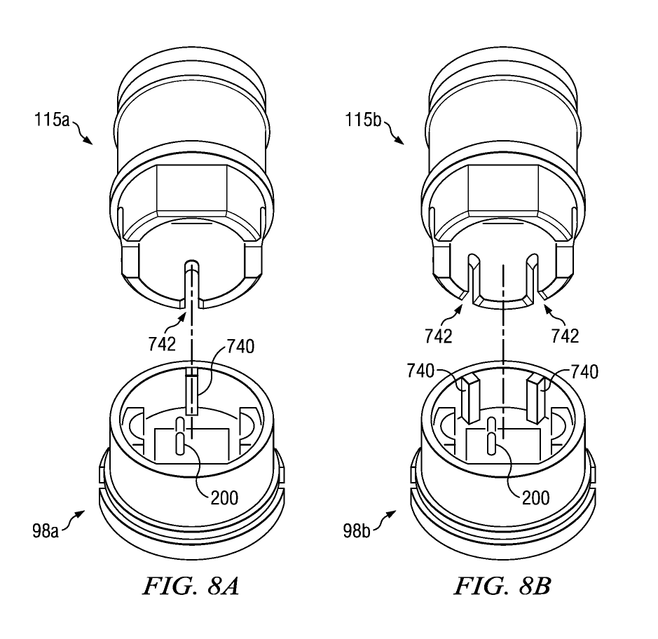

Air supplied to inlet air ducting 114 may be ambient air from an air source 69. The air source 69 may be a compressed air source that may incorporate a fan, a blower or a compressor that may be external or internal to the hand held spray member 101. The compressor of the air supply may be any suitable air moving device, such as a fan, blower, turbine, or piston, rotary or diaphragm compressor, or other air pump. Air from the air source 69 flows to the air heating system 117, which then heats the received air as it passes to the nozzle 104. In certain embodiments, the air source 69 may increase the temperature of the air slightly (as shown in the FIG. 8E graph). With this temperature increase, a lower rated heating system 117 may be used. In any event, the air received by the nozzle 104 is warmer than the ambient air temperature (i.e., warmer than the air temperature where the target 12 is located).

Input to power and control the air source 69, the air heating system 117, and the liquid source 110 may originate at the base unit 70. For example, a control and/or power line may run from the base unit 70 to the heating system 117. As explained in more detail below, this line may run with an air hose that also runs from an air source 69, which may be in the base unit 70, to the heating element 117.

A schematic representation of an example embodiment of the base unit 70 is shown in FIG. 3. The base unit 70 may comprise any suitable electrical, mechanical, or electro-mechanical control system that is responsive to user actuation to control operation of the hand held spray member 101 in support of the various operating modes described herein. After a user switches a power switch 720 to the “On” selection, the base unit 70 may control the liquid flow and/or the supply of liquid. The base unit 70 may also control the delivery of heated air for output at the nozzle 104. It may also control operation of the heating system 117 so as to control the temperature of the heated air. The base unit 70 may also be portable. In certain embodiments the base unit 70 may include wheels that enable easy transport. In other embodiments a smaller sized base unit may be carried on a person's shoulder or back like a backpack or shoulder pack.

An air control output 702 of the base unit 70 may modulate power to control the air source 69 or may send signals or otherwise communicate with the air source 69. Thus, a user providing input at the base unit 70 may control the rate of flow of the air to the spray member 101. The air source 69 may be any suitable voltage. For example, it may be 115 or 230 Volts AC. In certain embodiments, a jumper may allow the voltage to be switched from one voltage setting to another voltage setting. Furthermore, the base unit 70 may sense operating conditions of the system through a collection of input circuits. For example, an air sensor input 710 may be located in the air ducting and may sense reduced or absent air flow or an over pressure situation. These two situations may result in the event of a punctured or blocked air hose, respectively. A current transformer 703, coupled between a heater control output 706 and the heating system 117, may be employed to sense and interrupt power to the air heating system 117 to prevent overheating. The air control input 710 may sense a lack of air flow to ensure that the air source 69 is operational and air is flowing to the spray member 101 before power is applied to the heating system 117. This may prevent a dangerous overheating condition that may occur if the heating system 117 was to heat without air flowing through it.

The air source 69 may be operable at any suitable power, such as up to 1.5 horsepower. A user may select the power level using air switch 716. The air switch 716 may allow operation at increasing speeds of the air source 69. For example, the air source 69 may operate at 70% and 85% of full power, and at full power. The performance of the air source 69 may also be controlled by logic embodied in software instructing the operation of the air source 69.

According to an embodiment of the present disclosure, a liquid control output 704 may control the rate of flow of liquid emitted at the nozzle 104. The liquid control output 704 may power and communicate with the liquid source 110. In certain embodiments, the liquid source may be external to the spray member 101, and in some embodiments, the liquid source may be part of the base unit 70. Through the liquid control output 704, an operator may use the base unit 70 to control the amount of liquid that is included in the spray mist 33. In other embodiments, liquid flow may be controlled by a mechanical valve system within the hand held spray member 101. At the very least the state (on/off) of passage of skin treatment liquid to the spray jet outlet 105 of the nozzle 104 may be controlled. In addition, a rate of flow of skin treatment liquid may also be controlled. In either case, the skin treatment liquid is atomized at the spray jet outlet 105 to form the spray cloud 33.

The base unit 70 may also include a heater control 706. The heater control 706 may provide power and communicate with the air heating system 117. The air heating element 117 may also have increasing operating power selections. In one embodiment, the heating system 117 may supply power to a 720 watt heating element that may run at 30% and 70% of full power, and at full power. According to the teachings of the present disclosure, a resistance heater 116 may operate in the range of 0 to 1600 Watts. The output power may be modulated by a TRIAC circuit or other modulating circuit known in the art. In zero Watt operation, the air may be heated by the air source 69 alone. An electrical cable 705 may run from the base unit 70 to the heating system 117. The electrical cable 705 may electrically couple components of a heater circuit that allows the heating system 117 to be powered and controlled. The electrical cable 705 may transmit power and/or electrical control signals between the base unit 70 and the heating system 117. In certain embodiments, the electrical cable 705 from the heater control 706 to the air heating system 117 may follow or be contained within a hose 78 that extends from the air source 69 to the air heating system 117. According to other embodiments, the power wire from the heater control 706 to the air heating system 117 may be separate from the hose 78.

Certain safety features may be associated with the power supplied to the air heating system 117. For example, a current transformer 703 may be electrically coupled between the heater control 706 and the air heating system 117. The current transformer may sense when there is an electrical disconnection from the air heating system 117. In the event this electrical disconnection is detected by the current transformer 703, power to the air heating system 117 may be shut off almost instantly. This may prevent a live electrical wire from being dangerously exposed if an unintentional and unexpected break in the electrical connection occurs. Moreover, additional safety may be provided because the electrical connection between the heater control 706 and the air heating system 117 may be in the form of a keyed connector. According to this embodiment, only the proper voltage heater control 706 may be connected with the proper voltage air heating system 117. This may eliminate a dangerous condition by preventing a higher voltage heater control 706 from being mistakenly connected to the air heating system 117. The air heating system 117 may also include a thermal switch 119 and/or a thermal fuse 118 to detect malfunction, improper operation or dangerous conditions in the air heating system 117 itself. Opening and closing of the thermal switch 119 and/or thermal fuse 118 may be detected by the CT 703 and appropriate system control or warning function may be performed by the base unit 70.

A remote operation input 708 may be part of the base unit 70. The remote operation input 708 may be separate from or part of the base unit 70. According to certain embodiments, the remote operation input 708 may require a certain condition to be satisfied before the base unit 70 and consequently the spraying system 10 is operational. For example, the remote operation input 708 may allow the spraying system 10 to be operational if a credit card payment transaction is conducted, or a particular magnetic card is read. In certain embodiments, an Internet connection that may be password protected may communicate with base unit 70 through the remote operation input 708 and may only allow operation if a particular password is received. The remote operation input 708 may also be associated with a timer that only allow the spraying system 10 to be operational for a predetermined period of time. Once the particular condition is satisfied, the remote operation input 708 communicates with the base unit 70 to allow it to become operational. This communication may be in analog or in the form of digital signals, such as serial signals transmitted over a wireline or wirelessly. In other embodiments, the remote operation input 708 may allow the base unit 70 to be operational if a certain radio frequency identification (RFID) signal is received.

A trigger input 712 may be incorporated with the base unit 70. The trigger input 712 may allow the spray member 101 to communicate with the base unit 70. This may be important to allow the user to control functionality of the base unit while performing the spraying application. In certain embodiments, the trigger input 712 may allow proportional liquid, air, or heat control. Thus, when the trigger of the spray member 101 is actuated the user may control air flow, liquid flow, heater intensity, and the like.

The base unit 70 may also alert a user or operator that a fault condition has occurred through the use of fault indicators 714. In certain embodiments, the fault indicators 714 may be in the form of a lamp (LED, LCD, etc.) that illuminates when a fault condition has occurred. For example, if the base unit 70 senses a malfunction of the air source 69, the fault indicator 714 corresponding to the air source 69 may illuminate.

In other embodiments, fault indication and general system parameters may be displayed and controlled by a computer system 76. The computer 76 may be any type of computing device that receives input data, processes that data through computer instructions in a program, and generates output data. Such device may be a hand-held device, tablet, laptop or notebook computer, desktop computer, minicomputer, mainframe, server, mobile phone, smart phone, personal digital assistant, other device, or any combination thereof. According to an embodiment, the base unit 70 may detect and communicate signals to the computer 76 that correspond to operating conditions of the spraying system 10, such as temperature, air flow, liquid flow, electrical problems (shorts, opens, over-current, over-voltage, power, ground faults, error messages, and the like). The computer 76 may also save information regarding the system in memory. For example, the computer 76 may store data concerning fault events and a time stamp as to when each fault occurred. Other parameters and an associated time stamp saved by the computer 76 may be, power on/off condition, air temperature and pressure data, a particular skin treatment liquid preference of a customer, and a particular customer's credit card or other payment information received by the system. All of the information recorded and stored by the computer 76 may be stored in a database and correlated to related information. In certain embodiments, specific information may be organized and stored for each particular customer. All of the data stored in the computer 76 may be displayed, printed, communicated to a separate associated communication device, and the like. In certain embodiments, the computer 76 may display or print a fault report providing information about the specific operation parameters of the system over a predetermined period.

The computer 76 may communicate with the base unit 70 and other computing devices through a communications network. Generally, the communication network provides for the communication of packets, cells, frames, or other portions of information between the computer 76 and the base unit 70. The computer 76 may receive and transmit packets or other signals to the base unit 70. For example, the computer 76 may communicate packets through a packet-switched communications network, such as the Internet. The communication network may be any network capable of transmitting data or messages. It may be implemented as a local area network (LAN), wide area network (WAN), global distributed network such as the Internet, an intranet, extranet, or any other form of wireless or wireline communication network.

In certain embodiments, the support, enclosure or housing configuration 101 may support an electrostatics system (not shown) coupled to the nozzle 104 which, in a preferred implementation, inductively charges the spray cloud 33 output from the nozzle 104. The electrostatics system may receive power from the same external power supply which supplies power to the heating system 117, such as the base unit 70. It will be understood, however, that other forms of electrostatic charging may be implemented, including contact charging in which the electrostatic charge is applied to the liquid which is received by the hand held spray member 101. In addition, the spray cloud 33 may be ionically charged by an ionizing device located in the air stream. The ionizing device may be upstream of the nozzle 104 or it may be attached to the hand held spray member 101 downstream of the nozzle 104. Electrostatically charging the spray cloud may improve coating uniformity and reduce overspray.

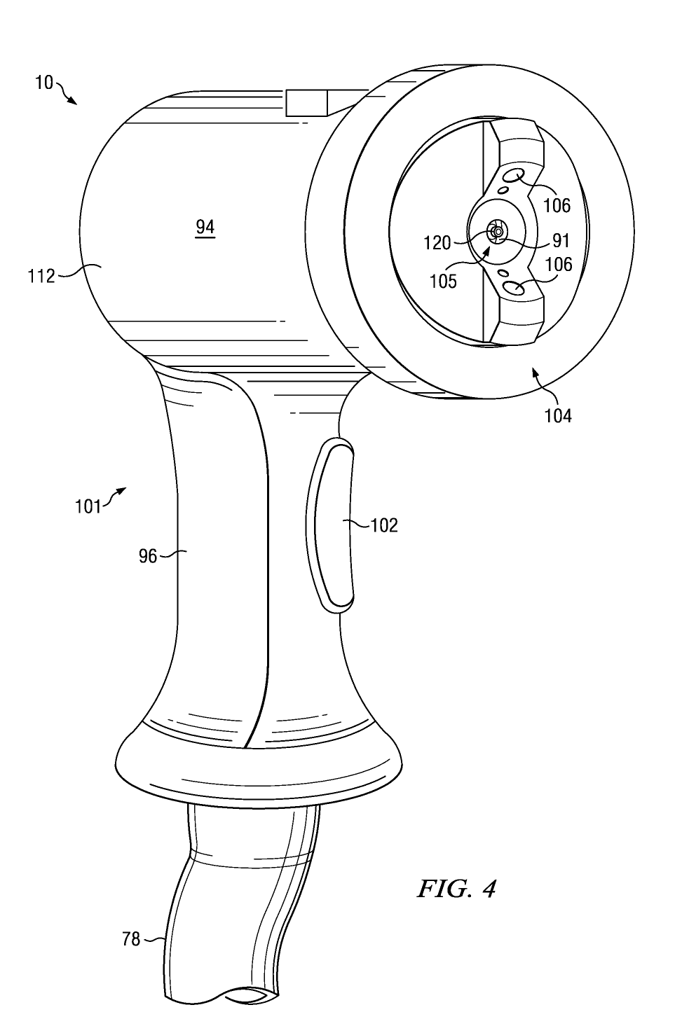

Reference is now made to FIG. 4, which illustrates an exemplary implementation of a hand held sprayer of the type represented in FIG. 1. The support, enclosure or housing configuration 101 of the hand held spray member implementation includes a suitably sized and shaped housing (or shroud) 112 for containing the nozzle 104, ducting for air and liquid flow, control device 52 for controlling liquid flow, air heating system 117, and a trigger-type 102 actuator for controlling operation of the hand held spray member. In an exemplary configuration, the housing 112 includes a barrel shaped portion 94 and a handle shaped portion 96. The spray member 101 may be configured to receive and connect to a hose 78. The hose 78 may run from the air source 69 to a fitting on spray member 101. As previously described, the hose 78 may be a conduit that carries air and/or liquid to spray member 101, where it may be converted into spray mist 33.

The front of the enclosure or housing configuration 101 of the hand held spray member implementation shows the spray nozzle 104 of the air atomizing type with the spray jet outlet 105 and mist shaping air ports 106 provided immediately adjacent the nozzle spray jet outlet 105. According to certain embodiments, the spray jet outlet 105 may include the liquid tip body 120 and an atomizing air port 91 that annularly surrounds and is concentric with the liquid tip body 120. Concentric liquid and air ports may be particularly suited for spray applications used to coat a target surface 12. The mist shaping air ports 106 supply air used by the nozzle 104 for pattern shaping of the spray cloud, for example, to shape the spray cloud into a flat fan-like spray shape, which may allow greater spray coverage of the target surface. In other embodiments, the spray cloud may be shaped to form a pinpoint spray pattern, which may allow more spray to coat a smaller portion of the target surface.

In certain embodiments, adjustable porting may allow additional control over the mist shaping air. A port size may be adjusted by rotating an air cap to a position that blocks air from escaping through a portion of the pattern shaping ports 106. This blockage will cause higher pressure air through the atomizing port 91 or other heated air emitting orifices. Adjusting porting may allow the atomizing air port 91 to supply relatively higher pressure air used by the nozzle 104 for atomization of the spray liquid to create the spray cloud. This air pressure may be higher than the air pressure used for pattern shaping The air pressure in the nozzle may be less than in conventional liquid spray systems such that less heat of the pressurized air may be lost due to expansion as it leaves the nozzle 104. In certain embodiments, the pressure may be less than 10 psi.

Air flow ports and outlets may be enlarged to reduce the expansion cooling effect on the heated air. The volumetric flow rate for the heated air emitted by the nozzle 104 and the air port 91 may be in the range of 3 to 50 standard cubic feet per minute (SCFM). The air pressure may be between 0.3 to 30 pounds per square inch (psi). In order to minimize heat loss due to expansion, the air pressure at the nozzle 104 may be limited to less than 5 psi. In certain embodiments, the nozzle pressure may be between 0.2 and 1 psi.

The physical embodiment of the housing illustrated in FIG. 4 for the enclosure or housing configuration 101 of the hand held spray member implementation is exemplary in nature, it being understood that any suitable industrial design for the housing could be used. The design is capable of being hand held and further support a suitably positioned trigger-type 102 actuator on the outside surface of the housing. The illustration in FIG. 4 of a traditional gun-shaped housing design with a barrel and handle for the hand held spray member is not to be considered as critical or limiting.

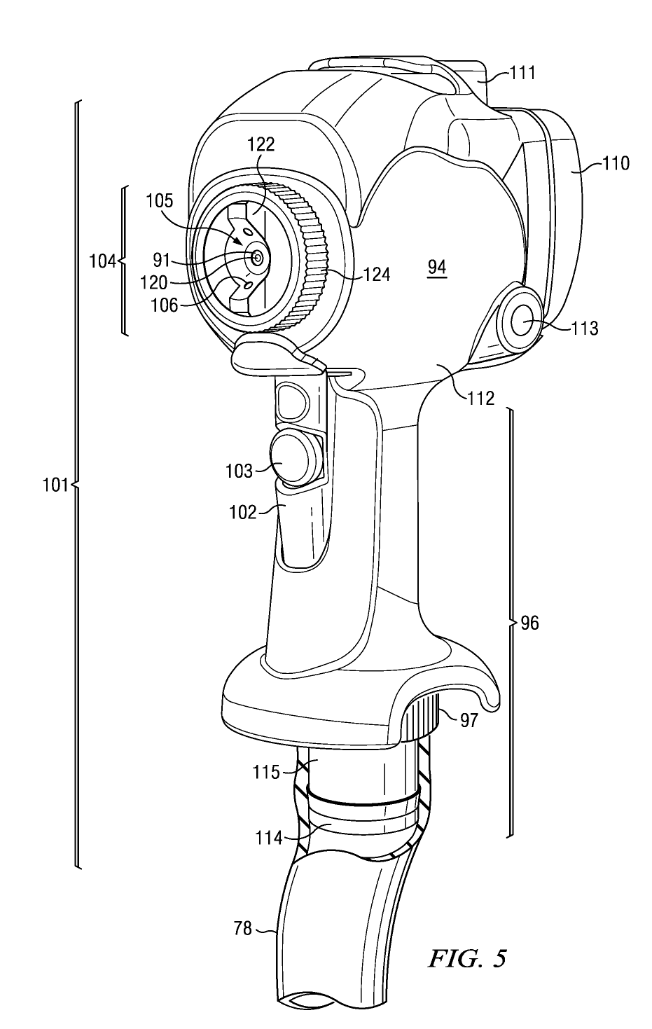

Reference is now made to FIG. 5 which illustrates an exemplary implementation of a hand held sprayer of the type shown in FIG. 1 and having a similar configuration to that shown in FIG. 4. This external view shows the housing (or shroud) 112, the nozzle 104, spray jet outlet 105, main atomizing air port 91, and pattern shaping air ports 106. The nozzle 104 includes an air cap 122 that is secured to the hand held sprayer 101 by a spray jet retaining ring 124. The air cap 122 me be made of any suitable material. In certain embodiments, it may be metal or plastic. The air cap may channel heated air to the atomizing air ports 91 and the pattern shaping air ports 106.

The liquid source 110 is attachable to a rear of the barrel portion 94 of the sprayer. The liquid source 110 comprises a single liquid tank supplying a single type of liquid for spray application. The tank may be filled through a cap 111. The container forming the liquid source 110 is also detachable through actuation of a mechanical release button 113. This allows the user to change the type of spray liquid being applied by changing liquid containers. The inlet air ducting 114 is provided at a base of the handle portion 96. Tubular member 115 supports connection of the air hose 78 to the hand held sprayer 101 using the retention ring 97. The sprayer 101 also includes an external trigger 102. The limit of trigger 102 actuation may be controlled by a set screw 103.

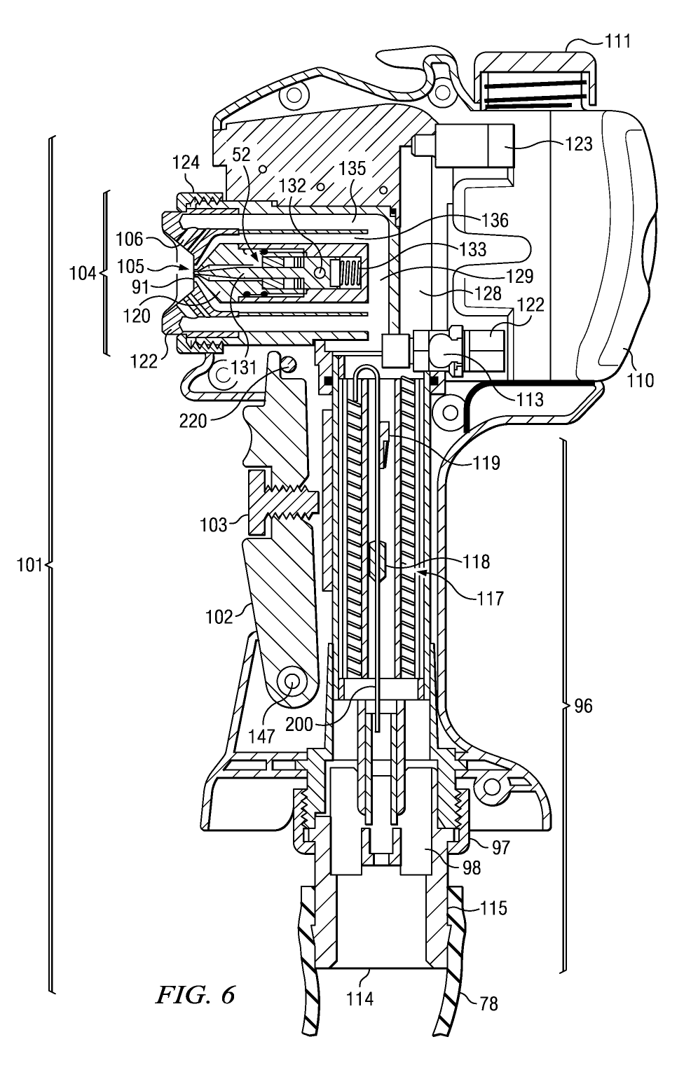

Reference is now made to FIG. 6, which illustrates a cross sectional view of the hand held sprayer shown in FIG. 5. The nozzle 104 used in this implementation is of an HVLP type, but could comprise any air-assisted nozzle having an air flow and creating the spray cloud. Liquid for spraying is passed from liquid valve 52 by internal ducting to the nozzle spray jet outlet 105 where it is atomized in response to the air supplied at the atomizing air port 91 to form the atomized spray cloud and pattern shaped in response to the air supplied at the air ports 106 so as to shape the atomized spray cloud (for example, into a fan-like pattern). Heated air is passed by internal ducting and distributed among and between the air ports 91 and 106.

In an alternative configuration, the air ports 106 may be configured to not only shape the atomized spray cloud but also to provide heated air for purposes of warming the spray cloud. To implement this configuration, the internal ducting of the nozzle 104 may be configured so that the pattern shaping air ports 106 receive the heated air. Additionally, the pattern shaping air ports 106 may be designed to be low pressure outlets that minimize a nozzle cooling effect on the spray cloud.

Air is communicated through the hose 78 and received at the inlet air ducting 114 at the base of the handle portion 96. The received air passes up through the handle portion 96. The air heating system 117 may be located at various locations in the hand held sprayer 101. For example, the heating system 117 may be installed in the handle portion 96 within the ducting carrying the air received at inlet ducting 114. The hose 78 may thread into the tubular member 115 and be further secured to the handle portion 96 using the retaining ring 97. In other embodiments, the heating element 117 may be located in the hose 78 and/or in the tubular member 115 proximate the hose end that connects to the hand held spray member 101. In this embodiment, the hose 78 including the heating element 117 may be detachable such that the heating element 117 may be removed from the spray member 101. Regardless whether the heating element 117 is removable or non-removable from the spray member 101, locating the heating element 117 near the spray member end of the hose 78 may facilitate heat retention because heated air is not required to flow through a significant length of hose 78, such that considerable heat is lost. In certain embodiments, the hose 78 may also carry the wires to make an electrical connection between the base unit 70 and the spray member 101. In this embodiment, an electrical connector 98 may be located within the handle portion 96. However, if the heating element 117 is removable with the hose 78, then it may not be necessary to have an electrical connection between the hose 78 and the hand held spray member 101.

Embodiments of the electrical and air hose connection are shown in FIGS. 8A and 8B. Embodiments of an electrical connector 98a and 98b may allow a three-way keyed connection to the hose 78. A D-shaped connector or tubular member 115a and 115b may be inside the hose 78 and may connected to the electrical connector 98a and 98b. The electrical connection to provide power to the heating system may be made through power lines 200 of the heating system 117. Prongs 740 protruding from the electrical connector 98a and 98b may correspond to slots 742 in the tubular member. When the appropriate number of prongs are mated with the appropriate number of slots and the D-connector is oriented properly, it may be ensured that only the proper voltage base unit 70 may be connected to the spray member 101. The electrical connection allows the heating element 117 to receive power and emit thermal energy. In certain embodiments, air may flow through the hose 78, enter the hand held spray member 101, and flow around the power lines 200.

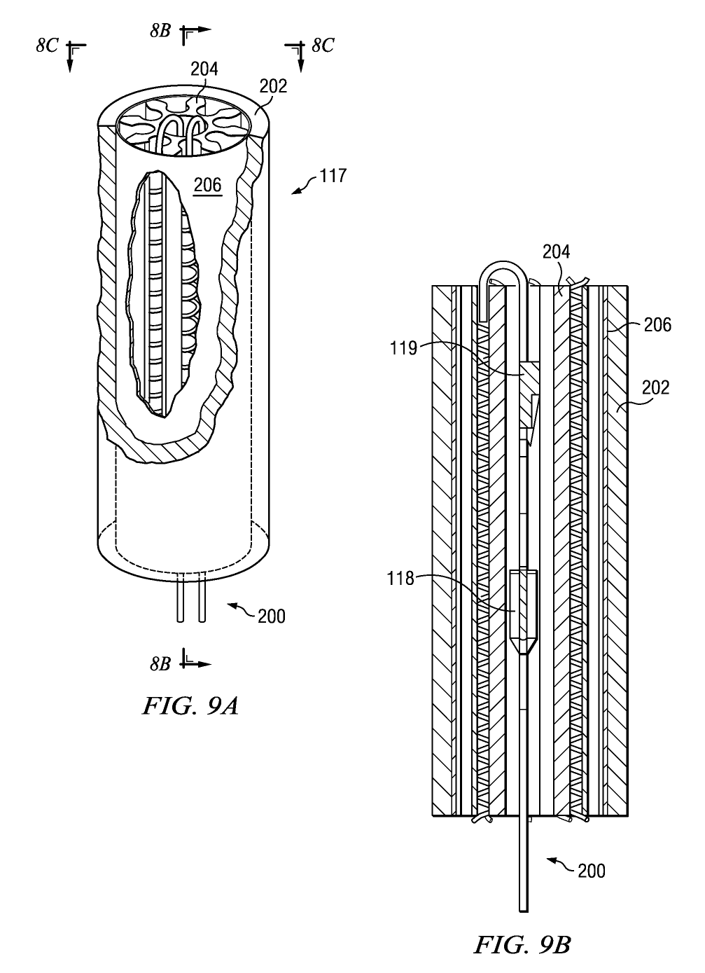

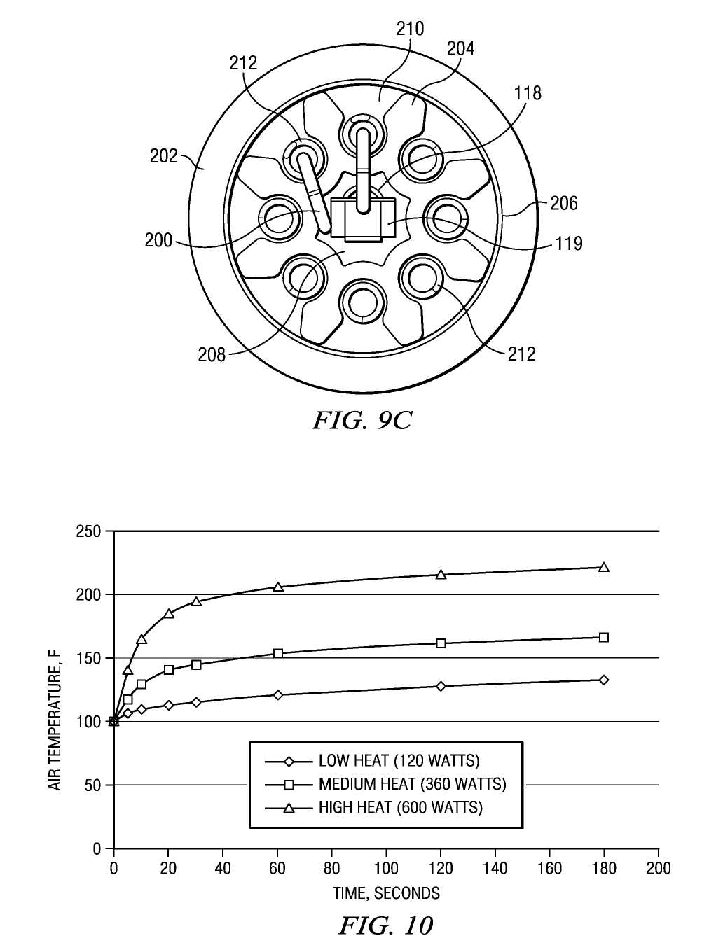

The heating system 117 includes a thermal fuse 118 and a thermal switch 119 (in the form, for example, of a thermostat) functioning as safety devices with respect to sprayer operation so as to protect against an overheating or malfunctioning situation. A perspective, partially broken away view of the heating system 117 is shown in FIG. 9A. A longitudinal cross-section is shown in FIG. 9B. A lateral cross-section is shown in FIG. 9C. Power to the heating system 117 is supplied by power lines 200. The heating system 117 includes a cylindrical tube support 202. The cylindrical tube support 202 may be made from an electrically and thermally insulating material such as Garolite or other suitable fiberglass composite or plastic material. A ceramic core 204 is installed within the tube support 202. A mica wrap 206 is positioned between the inner surface of the tube support 202 and the outer periphery of the ceramic core 204. The ceramic core 204 is formed to include a central longitudinal channel 208 and a plurality of peripheral longitudinal channels 210. These channels 208 and 210 are sized to permit the flow of air through the heating system 117. The power lines 200 pass through the central longitudinal channel 208, and the thermal fuse 118 and thermal switch 119 are installed within the central longitudinal channel 208. A coiled resistance wire 212 is installed within each one of the peripheral longitudinal channels 210. The coiled resistance wires 212 are electrically connected to each other and to the power lines 200.

The heating system 117 is designed to quickly ramp up to a desired air heating temperature and maintain that temperature over the course of a spray session. In addition, if the heating system 117 gets too hot (for example if there is no air flowing through the heating system 117), the thermal switch 119 may operate to interrupt the power to the heating system 117 to prevent a dangerous overheating condition. The thermal switch 119 may be a resettable thermal switch. As such, the thermal switch 119 may interrupt the power substantially immediately, without requiring communication of a signal to the base unit 70. However, a signal may still be communicated to the base unit 70 indicating the overheating condition and the base unit 70 may respond accordingly.

FIG. 10 is a graph showing air temperature at the output of the heating system 117 for three different power configurations (low, medium and high). The graph shows how temperature quickly rises from an ambient (or near ambient) air temperature to an elevated temperature level. In a preferred operational scenario, the heating system 117 is controlled on initial start up of the spray system in the high power configuration so as to achieve fast temperature rise time. The system may then switch operational power to a reduced medium or low power level depending on user desire and comfort. In certain embodiments, the heating system may be electrically and/or thermally insulated to protect the operator from being burned or receiving an electrical charge.

Reference is once again made to FIG. 6. After passing through the heating system 117, the air (now heated air) passes through internal ducting and is made available within the hand held sprayer 101 for a number of purposes. First, the heated air is delivered to an air channel 128, which, in certain embodiments may be coupled through an inlet check valve 123 to the liquid supply 110 container. The check valve 123 only permits air to enter the liquid supply 110 container, and thus the air supplied from the air channel 128 functions to pressurize and heat the liquid supply 110 container. In other embodiments, the liquid supply 110 may not be pressurized by the heated air. For example, in one embodiment the liquid supply 110 may be external to the hand held spray member, and the heated air may not reach the liquid supply 110.

Second, the heated air is delivered to a nozzle air channel 129. In certain embodiments, the nozzle air channel 129 may be separate from the air channel 128. This nozzle air channel 129 is coupled to the pattern shaping air ports 106 through pattern shaping air channel 135. Thus, heated pattern shaping air is supplied to the pattern shaping air ports 106 of the nozzle 104. This nozzle air channel 129 is further coupled to the air atomization ports 91 through atomization air channel 136. Thus, heated atomizing air is also supplied to the air atomization air ports 91 at the spray jet outlet 105 of the nozzle 104. One or more air valves (not explicitly shown) may be used to control heated air delivery and the air pressure to the atomizing air port 91 and the pattern shaping air ports 106.

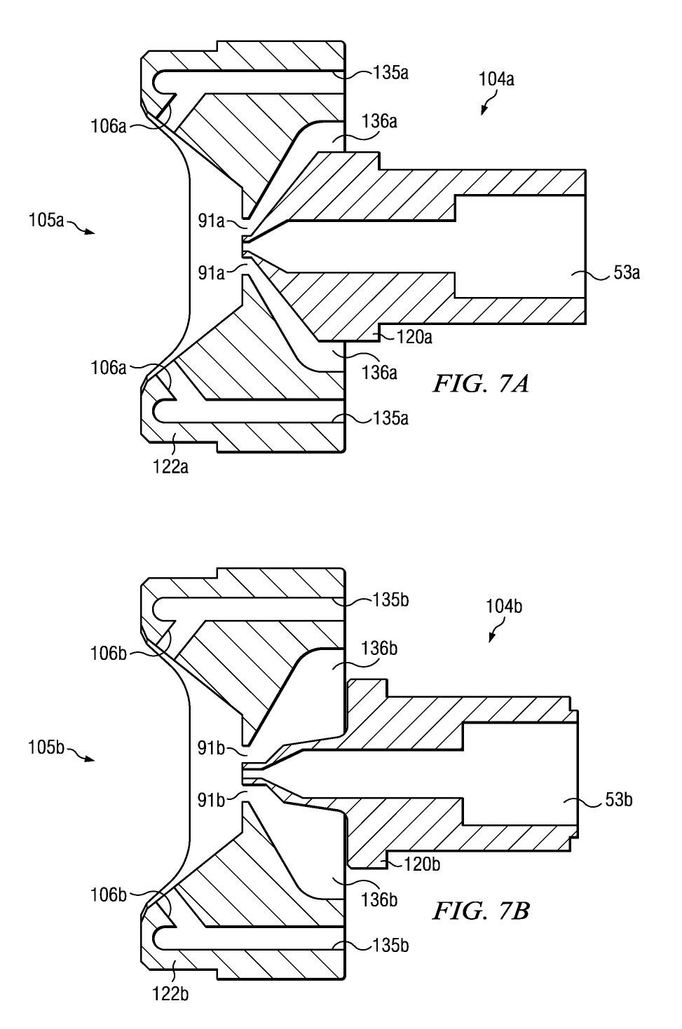

Simplified diagrams of embodiments of the nozzle 104 of FIG. 6 are shown in FIGS. 7A and 7B. The nozzles 104a and b are shown without the details of the liquid control valve 52 or the spray jet retaining ring 124. Embodiments of the liquid tip body 120 may have any suitable cross sectional diameter or thicknesses. For example, the liquid tip body 120b may have a reduced cross sectional thickness and cross sectional diameter than that of liquid tip body 120a. As a result, the atomization air channel 136b may be larger than the atomization air channel 136a. This reduced thickness of the liquid tip body 120b may allow the heated atomization air to heat the liquid flowing through or contained in the liquid tip body 120b faster and hotter than the same heated atomization air would heat the liquid flowing through or contained in the liquid tip body 120a. In certain embodiments, the liquid tip 120b may have an approximately 60% reduction in thermal mass over the liquid tip body 120a.

For each nozzle embodiment, heating of the liquid occurs as the atomization air channel 136 receives heated air from the heating system 117 through the nozzle air channel 129, the heated air will also heat the liquid tip body 120 as the heated air flows to the atomizing air ports 91. This heated liquid tip body 120 may transfer heat to the liquid as it flows to spray jet outlet 105. The liquid tip body 120 may be metal or other material that effectively conducts heat. In one embodiment, the liquid tip body 120 may be stainless steel.

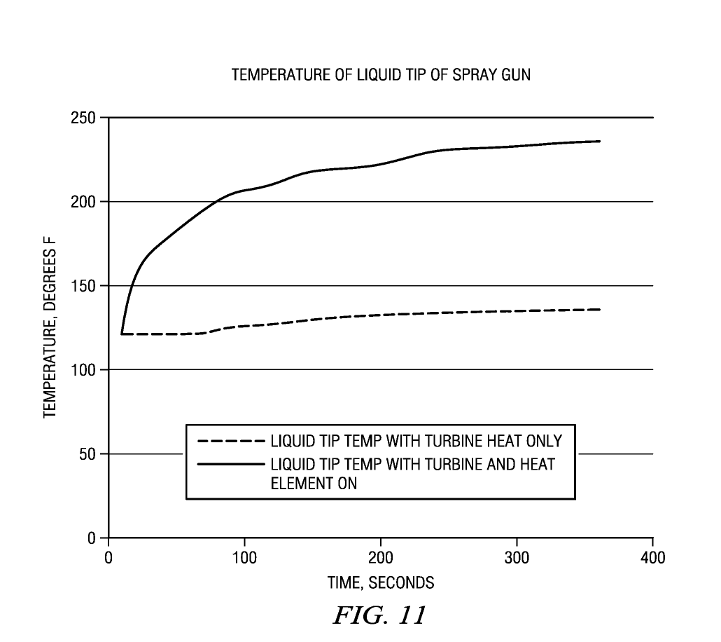

FIG. 11 illustrates the temperature of the liquid tip body 120 as heated air flowing through the spray member 101 causes it to heat up. As shown, the heated air may allow the liquid tip body 120 to reach temperatures of approximately 225 degrees Fahrenheit. FIG. 11 also illustrates a baseline temperature of the liquid tip body 120 when the heating element 117 is not operating, but the air is being heated by the air source/air turbine 69. The testing conditions included attaching the hand held spray member 101 to an external air source using a hose that was approximately 10 feet in length. Under these conditions, the turbine of the air source 69 was allowed to run for between 5 and 10 minutes to reach the baseline temperature shown. Thus, it can be interpolated from FIG. 11 that the heating system 117 causes a significant rise in temperature of the liquid tip body 120 and that elevated temperature may be reached relatively soon after initial start-up. The heated liquid tip body 120 may, in turn, transfer heat to the liquid and allow more uniform emission of the liquid from the spray jet outlet 105.

Heated air exiting from the air atomization port 91 may assist atomization of the liquid provided from the liquid supply 110 container and passing through the quick connect valve 122 and internal ducting to the nozzle spray jet outlet 105 to form the spray cloud 33. In certain embodiments, the air pressure may be reduced such that the spray mist remains warm. For example, air pressure below 10 p.s.i. may create an effective spray mist and reduce the amount of heat loss due to expansion of the air as it exits the atomizing air port 91. In certain embodiments, nozzle geometry may reduce heated air cooling due to rapid air expansion at the nozzle 104. Corresponding to the reduced air pressure, the flow rate of the liquid may also be reduced to allow for atomization of a lesser quantity of liquid to ensure that all of the liquid ejected from the spray jet outlet 105 is atomized.

The liquid for the spraying operation is sourced from the liquid supply 110 container. The liquid in the liquid supply 110 container is coupled through an outlet quick connect valve 122 through internal ducting (not explicitly shown) to the nozzle spray jet outlet 105. The outlet quick connect valve 122 for the liquid supply 110 container in this implementation does not function to control the state or rate of liquid flow or the size of the atomized spray cloud. Rather, a separate liquid flow control device or liquid valve 52 is provided in the nozzle 104. This liquid control valve 52 in the illustrated configuration comprises a needle valve (to be described) associated with the nozzle jet outlet 105. In other embodiments, the flow of liquid may be controlled by a pump, a remote solenoid valve, or a pneumatically controlled valve. The liquid flow control device may be internal to the hand held spray member 101 or may remote to the spray member 101. Also, the rate of flow of the liquid may be regulated by controlling air pressure into the liquid supply container 110 at inlet check valve 123.

When the liquid control valve 52 is closed, the flow of liquid from the liquid supply 110 container to the nozzle spray jet outlet 105 is blocked and only heated air may be delivered by the nozzle 104. As the liquid control valve 52 opens, liquid from the liquid supply 110 container flows to nozzle spray jet outlet 105. This flow may be assisted because the liquid supply 110 container has been pressurized by heated air passing into the liquid supply 110 container through the inlet check valve 123. In a non- needle valve implementation, the outlet check valve 122 may be configured to implement the functionality of the liquid control valve 52 (for example through controlling suction of liquid from the liquid supply 110 container to nozzle spray jet outlet 105).

In the needle valve configuration, the needle valve comprises a liquid flow needle 131 for the liquid control valve 52 that is biased by a spring 133 in a closed position that shuts off the flow of liquid to the nozzle spray jet outlet 105. The liquid flow needle 131 moves within the nozzle 104 in response to actuation of a pin 132. When the trigger 102 is actuated, the trigger mechanism rotates about the pivot 147 and engages the pin 220. Movement of the pin 220 (in response to the trigger 102 actuation) causes the control linkage mechanism to move the needle valve pin 132 and open the liquid control valve 52 by moving the liquid flow needle 131 within the nozzle 104. When the trigger 102 is in a fully released position, the control linkage mechanism (along with spring 133) sets the fluid flow needle 131 of liquid control valve 52 into a fully closed. As the trigger 102 is further actuated, the control linkage mechanism begins to open the needle valve. When the trigger 102 moves towards the fully actuated position, the control linkage mechanism sets the liquid flow needle 131 into a position where the liquid control valve 52 is fully open. The set screw 103 provides a mechanism for controlling the maximum degree of trigger 102 actuation and thus can limit the degree of opening the liquid control valve 52 in response to full actuation of the trigger 102.

The system 10 supports a controlled spraying and drying operation. The hand held spray member with the jet outlet 105 is used in one or more passes to spray heated air and a heated skin treatment liquid over the customer's skin. The heated air may be emitted through the atomizing air port 91 and/or the mist shaping air ports 106 to warm the spray cloud 33 produced by the nozzle 104. This heated air treatment serves two purposes: a) it dries the skin quickly after spraying which has been shown to enhance the end result of the spraying; and b) it keeps the customer warm (perhaps in anticipation of a subsequent spraying). The foregoing operations may then be repeated for those applications which require multiple spray passes (such as to provided thicker coverage or to change liquid application).

The system 10 described herein supports exercising control over the operation of the heated air flow, heat levels, nozzle operation, liquid selection, and nozzle movement.

Improved results using the apparatus and process described herein, with a trial using DHA (dihydroxyacetone) based sunless tanning compounds, include:
Increases tan color by allowing higher quantities of sprayed active ingredient to be deposited due to a layering process where the spray is applied; the skin is re-dried quickly by the warm air before another spray pass over the same target area;
Promotes deeper activity of DHA by drying the top layer of skin completely and possibly by drying inner layers of the stratum corneum skin layer; this results in longer lasting tan color;
Opens skin surface pores to allow for better penetration of tanning compound and skin care ingredients;
Reduces the occurrence of chill bumps on the skin that may result in an uneven and poor quality tan;
Properly controlled heated air dries the skin of any perspiration or other moisture, including the water based spray itself, that may cause an uneven tanning effect and prevent penetration into skin layers;
Prevents dripping or streaking of the sprayed material during the tanning process which can cause an uneven tanning result; and
Eliminates the step of drying the skin off with a towel which causes partial removal and disturbance of the evenly deposited layer from the spray application.

Although preferred embodiments of the method and apparatus of the present invention have been illustrated in the accompanying Drawings and described in the foregoing Detailed Description, it will be understood that the invention is not limited to the embodiments disclosed, but is capable of numerous rearrangements, modifications and substitutions without departing from the spirit of the invention as set forth and defined by the following claims.

1. Apparatus, comprising:
a hand held spray member having an interior portion defining an air channel, the air channel coupled to an external air source;
a heating unit disposed within the air channel and adapted to heat air flowing through the air channel;
a liquid tip body disposed within the air channel downstream of the heating unit, the liquid tip body adapted to receive a skin treatment liquid and to transfer heat thereto; and
a spray nozzle supported by the hand held spray member and including:
a liquid outlet adapted to emit the heated skin treatment liquid; and
an air outlet adapted to emit the heated air.

2. The apparatus of claim 1, wherein the emission of the heated skin treatment liquid and the heated air form an atomized spray cloud having a spray pattern for coating a target surface.

3. The apparatus of claim 2, wherein the spray pattern is either a pinpoint spray pattern or a fan spray pattern.

4. The apparatus of claim 1, wherein the spray nozzle emits an atomized spray cloud including the heated skin treatment liquid; and
the spray nozzle further comprises a pattern shaping port adapted to emit the heated air to shape a spray pattern of the atomized spray cloud.

5. The apparatus of claim 1, further comprising a hose coupling the hand held spray member to the external air source.

6. The apparatus of claim 1, further comprising an electrical connector, and wherein a power cord is removably coupled to the electrical connector, the power cord adaptable to supply power to the heating unit.

7. The apparatus of claim 6, further comprising an air hose coupling the hand held spray member to the external air source, the power cord being integral with the air hose.

8. The apparatus of claim 6, wherein the electrical connector is a keyed electrical connector.

9. The apparatus of claim 6, further comprising a base unit electrically coupled to the power cord, the base unit operable to detect an electrical disconnection of the power cord from the heating unit and in response interrupt power to the heating unit.

10. The apparatus of claim 1, further comprising a base unit operable to detect air flowing to the heating unit, and in response supply power to the heating unit.

11. The apparatus of claim 1, further comprising a trigger supported by the hand held spray member, and wherein flow of the skin treatment liquid is actuated by the trigger.

12. The apparatus of claim 1, wherein emission from the spray nozzle induces a drop in temperature of the skin treatment liquid due to a nozzle expansion effect, and wherein the emitted heated air counteracts the induced drop in temperature.

13. The apparatus of claim 1, wherein the spray nozzle is an air-assisted atomizing type spray nozzle.

14. The apparatus of claim 1, further comprising:
a container supported by the hand held spray member and adapted to hold the skin treatment liquid; and
wherein the container is configured to receive the heated air from the air channel and thereby increase a pressure within the container.

15. The apparatus of claim 14, wherein a valve couples the container to the air channel.

16. The apparatus of claim 1, wherein the spray nozzle emits an atomized spray cloud including the heated skin treatment liquid; and further including an electrostatic charge generator adapted to impart an electrostatic charge on the atomized spray cloud.

17. The apparatus of claim 1, wherein:
the hand held spray member defines a handle portion; and
the heating unit is positioned within the handle portion.

18. The apparatus of claim 1, wherein the heating unit further includes a core defining at least one longitudinal channel adapted to permit air to flow therethrough.

19. The apparatus of claim 18, wherein the at least one longitudinal channel is positioned in the center of the heating unit, and the core further defines a plurality of peripheral longitudinal channels adapted to permit air to flow therethrough.

20. The apparatus of claim 19, wherein each one of the plurality of peripheral longitudinal channels includes a respective coiled resistance wire.

21. The apparatus of claim 1, further comprising a thermal switch located within the air channel, the thermal switch operable to interrupt power to the heating element if a temperature of the thermal switch increases above a predetermined temperature.

22. The apparatus of claim 21, wherein the thermal switch is located within a longitudinal channel of the heating unit.

23. Apparatus, comprising:
a hand held spray member having a handle portion;
a heating unit within an air channel of the handle portion adapted to heat air;
a hose coupling the hand held spray member to an external air source; and
an air-assisted spray nozzle supported by the hand held spray member and including:
a liquid outlet adapted to emit a skin treatment liquid; and
an air outlet adapted to emit the heated air, the emission of the skin treatment liquid and the heated air forming an atomized spray cloud.

24. The apparatus of claim 23, wherein the spray nozzle further includes at least one pattern shaping port adapted to emit the heated air to shape a spray pattern of the atomized spray cloud.

25. The apparatus of claim 23, further comprising a liquid tip body proximate the liquid outlet and downstream of the heating unit, the liquid tip body being adapted to receive the skin treatment liquid, and wherein the heated air further heats the liquid tip body, and wherein the heated liquid tip body heats the skin treatment liquid.

26. The apparatus of claim 23, further comprising a heater circuit electrically coupled to the heating unit and being operable to interrupt power to the heating unit.

27. The apparatus of claim 23, wherein the heating unit further comprises a core that defines at least one longitudinal channel adapted to permit air flow therethrough.

28. The apparatus of claim 27, further comprising:
a thermal switch positioned in a flow of air from the external air source, the thermal switch operable to interrupt power to the heating unit if a temperature of the thermal switch increases above a predetermined temperature.

29. The apparatus of claim 28, wherein the thermal switch is electrically coupled to a current transformer, the current transformer being operable to detect the interruption in power.

30. The apparatus of claim 28, wherein the thermal switch is located in the at least one longitudinal channel.

31. The apparatus of claim 30, wherein the at least one longitudinal channel is positioned in the center of the heating unit, and the core further defines a plurality of peripheral longitudinal channels adapted to permit air flow therethrough.

32. The apparatus of claim 31, wherein each one of the plurality of peripheral longitudinal channels includes a respective coiled resistance wire.

33. A hand held spraying system, comprising:
a hand held spray member having a handle portion and a barrel portion, the hand held spray member being an air atomizing type sprayer;
a heating unit within an air channel of the hand held spray member, the heating unit adapted to heat air received from an external air source, the heated air flowing through the air channel;
a liquid tip body within the air channel downstream of the heating unit; and
a spray nozzle supported by the hand held spray member and including:
a liquid outlet adapted to emit a skin treatment liquid; and
an air outlet being annularly shaped and concentric with the liquid outlet;
the air outlet adapted to emit the heated air, the emission of the skin treatment liquid and the heated air forming an atomized spray cloud.

34. The hand held spraying system of claim 33, further comprising a trigger operable to allow the skin treatment liquid to be emitted from the liquid outlet.

35. The hand held spraying system of claim 33, wherein the heating unit is located within the handle portion.

36. The hand held spraying system of claim 33, wherein the heating unit is located within the barrel portion.

37. The hand held spraying system of claim 33, further comprising:
a hose coupling the hand held spray member to the external air source; and
wherein the heating unit is located within the hose proximate an end of the hose, the end of the hose configured to be attached to the hand held spray member.

38. The hand held spraying system of claim 33, wherein the spray nozzle further includes at least one pattern shaping port adapted to emit the heated air to shape a spray pattern of the atomized spray cloud.
